(19) United States
(12) Patent Application Publication
(10) Pub. No.: US 2018/0132821 A1
(43) Pub. Date: May 17, 2018

(54) ELECTROMAGNETIC TRACKER BASED ULTRASOUND PROBE CALIBRATION

(71) Applicant: KONINKLIJKE PHILIPS N.V., EINDHOVEN (NL)

(72) Inventors: EHSAN DEHGHAN MARVAST, NEW YORK, NY (US); SHYAM BHARAT, ARLINGTON, MA (US); AMIR MOHAMMAD TAHMASEBI MARAGHOOSH, RIDGEFIELD, CT (US); GREGORY COLE, OSSINING, NY (US); JOCHEN KRUECKER, WASHINGTON, DC (US)

(21) Appl. No.: 15/103,051

(22) PCT Filed: Dec. 16, 2014
(86) PCT No.: PCT/IB2014/066937

§ 371 Date: Jun. 9, 2016

Related U.S. Application Data
(60) Provisional application No. 61/917,615, filed on Dec. 18, 2013.
(60) Provisional application No. 61/917,615, filed on Dec. 18, 2013.

(51) Int. Cl. A61B 8/00; A61B 8/12

(52) U.S. Cl. CPC A61B 8/4254; A61B 8/12; A61B 8/587

(57) ABSTRACT

An ultrasound calibration system employs a calibration phantom (20), an ultrasound probe (10) and a calibration workstation (40a). The calibration phantom (20) encloses a frame assembly (21) within a calibration coordinate system established by one or more phantom trackers. In operation, the ultrasound probe (10) acoustically scans an image of the frame assembly (21) within an image coordinate system relative to a scan coordinate system established by one or more probe trackers. The calibration workstation (40a) localizes the ultrasound probe (10) and the frame assembly image (11) within the calibration coordinate system and determines a calibration transformation matrix between the image coordinate system and the scan coordinate system from the localizations.

The present invention generally relates to calibration of an ultrasound probe. The present invention specifically relates to localizing an ultrasound probe and an ultrasound image generated by the ultrasound probe within a same coordinate system for purposes of determining an otherwise unknown transformation matrix between the ultrasound probe and the ultrasound image.

Electromagnetic (“EM”) tracking of a position of an ultrasound image has many benefits in medical diagnosis and intervention. For example, during a prostate brachytherapy or biopsy, a transrectal ultrasound (“TRUS”) probe may be utilized for image guidance of a navigation of needles/catheters inside the prostate tissue to specific targets for the delivery of treatment thereto. More particularly, the EM-tracking the position of the TRUS probe is used for reconstruction of three-dimensional (“3D”) volumes and also for localization of other objects in the ultrasound image coordinate system.

In order to employ an EM-tracked TRUS probe, it is imperative to identify a relationship between the ultrasound image coordinate system and the EM-tracker coordinate system. Historically, the TRUS probes may be calibrated manually in a water tank. In this method, while the TRUS probe is immersed in water, a user inserts an EM-tracked pointed object (e.g., a needle) into the ultrasound field of view. As soon as the pointed object intersects with the TRUS image, the operator marks the position of the object tip on the ultrasound image. To achieve a reliable calibration, this position marking process is repeated several times and at several positions of the TRUS probe. However, manual probe calibration is subjective, tedious and time-consuming. Besides, most often, the object is advanced toward the ultrasound image from one side only. Therefore, the ultrasound image thickness reduces that accuracy of the calibration.

A calibration phantom with automatic calibration may solve the aforementioned problems of manual calibration.

The present invention proposes a method and apparatus for automatic calibration of a tracked ultrasound probe, particularly an EM-tracked TRUS probe.

One form of the present invention is an ultrasound calibration system employing a calibration phantom, an ultrasound probe (e.g., a TRUS probe) and a calibration workstation. The calibration phantom encloses a frame assembly within a calibration coordinate system established by one or more phantom trackers (e.g., EM trackers). In operation, the ultrasound probe acoustically scans an image of the frame assembly within an image coordinate system relative to a scan coordinate system established by one or more probe trackers (e.g., EM trackers). The calibration workstation localizes the ultrasound probe and the frame assembly image within the calibration coordinate system and determines a calibration transformation matrix between the image coordinate system and the scan coordinate system from the localizations.

Another form of the present invention is an ultrasound calibration system employing a calibration phantom and a calibration workstation. The calibration phantom encloses a frame assembly within a calibration coordinate system established by one or more phantom trackers (e.g., EM trackers). In operation, an ultrasound probe (e.g., a TRUS probe) acoustically scans an image of the frame assembly within an image coordinate system relative to a scan coordinate system established by one or more probe trackers (e.g., EM trackers). The calibration workstation localizes the ultrasound probe and the frame assembly image within the calibration coordinate system and determines a calibration transformation matrix between the image coordinate system and the scan coordinate system from the localizations.

An additional form of the present invention is an ultrasound calibration method involving a positioning of an ultrasound probe relative to a calibration phantom enclosing a frame assembly within a calibration coordinate system, an operation of the ultrasound probe to acoustically scan an image of the frame assembly within an image coordinate system relative to a scan coordinate system of the ultrasound probe, a localization of the ultrasound probe and the frame assembly image within the calibration coordinate system, and a determination of a calibration transformation matrix between the image coordinate system and the scan coordinate system as a function of the localizations.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

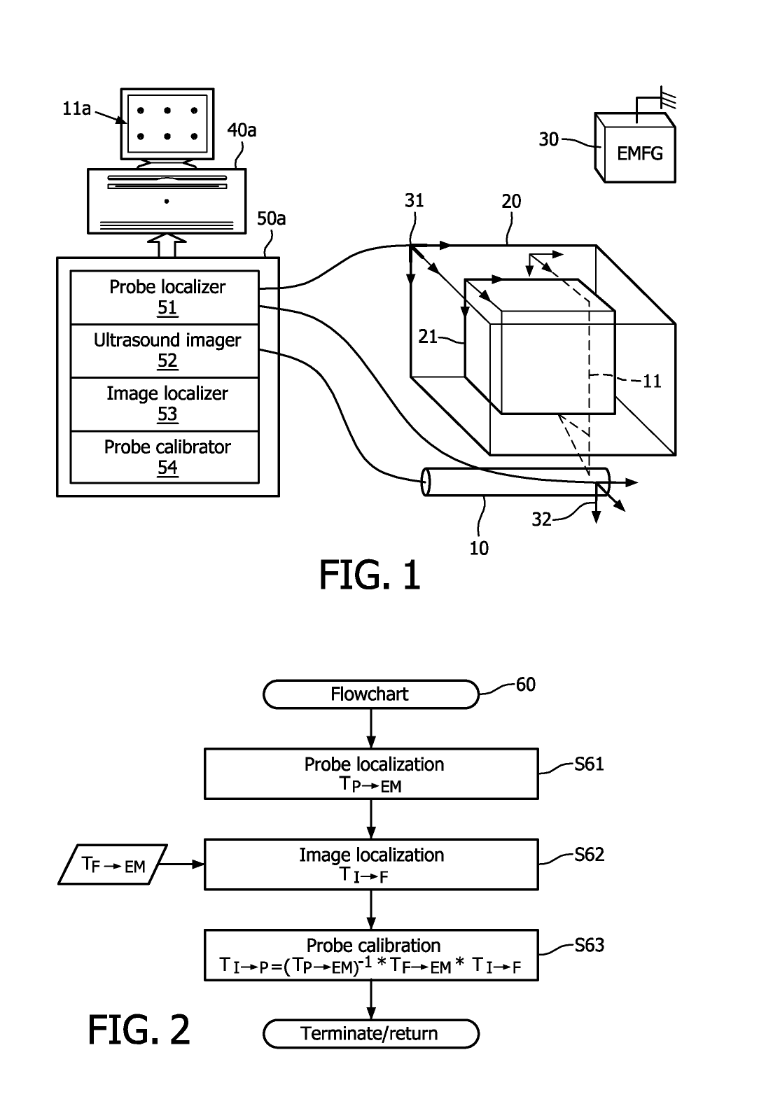

FIG. 1 illustrates an exemplary embodiment of an ultrasound calibration system in accordance with the present invention.

FIG. 2 illustrates an exemplary embodiment of an ultrasound calibration method in accordance with the present invention.

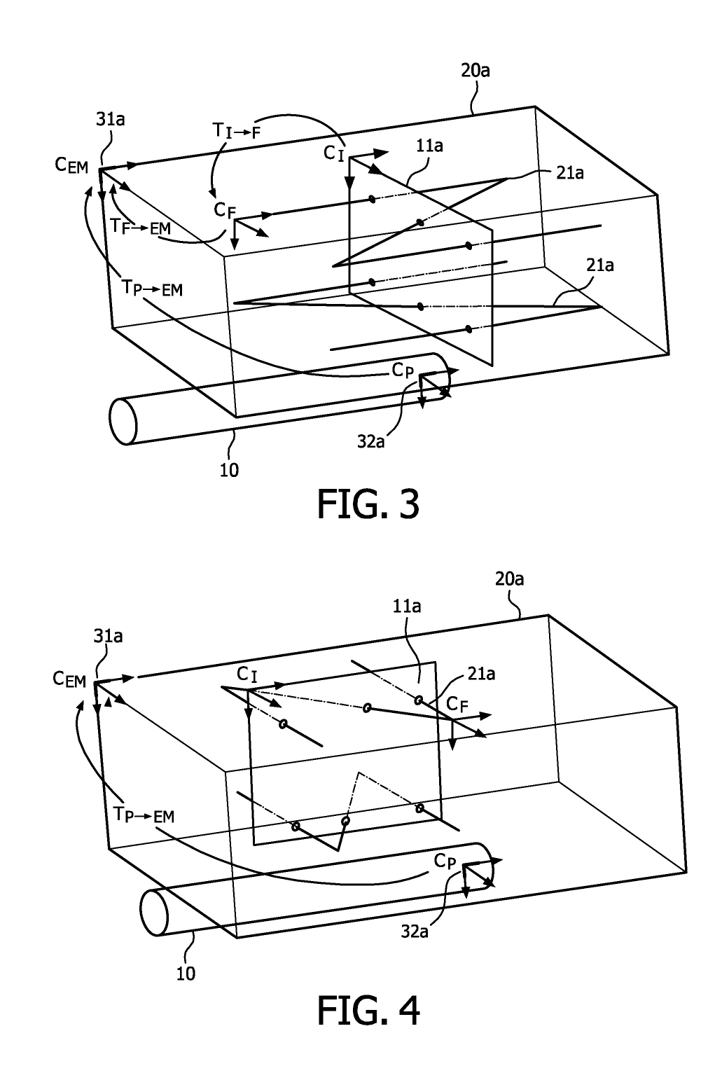

FIGS. 3-6 illustrate four (4) exemplary embodiments of a calibration phantom in accordance with the present invention.

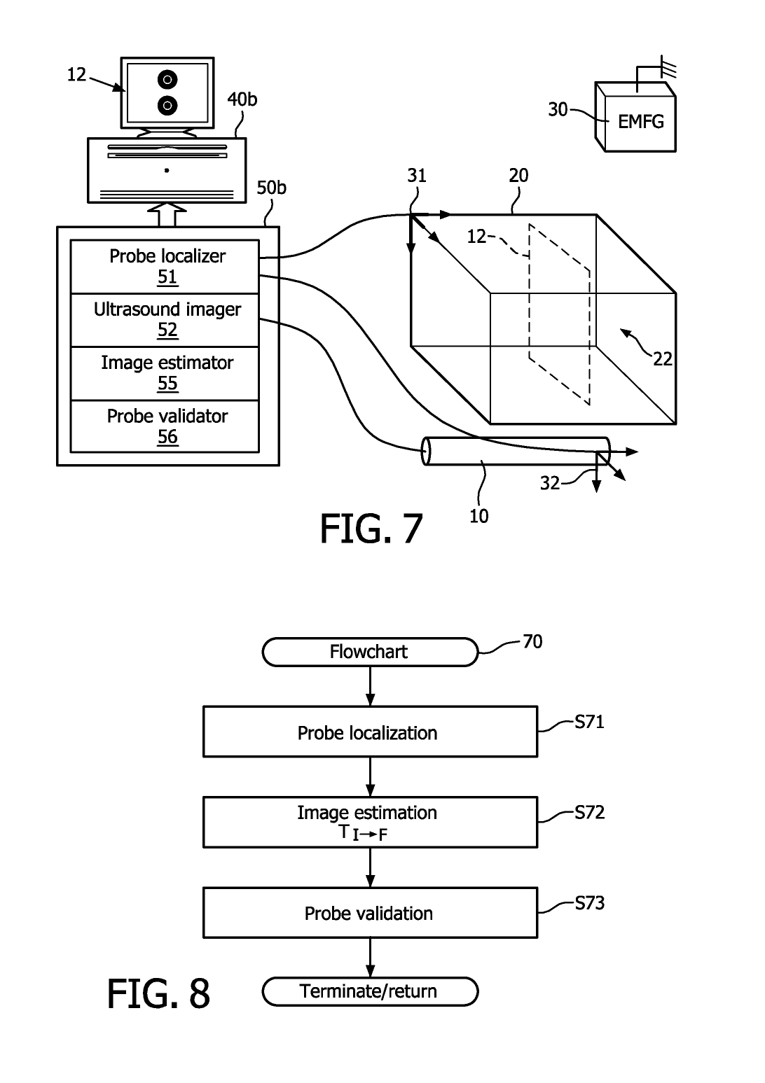

FIG. 7 illustrates an exemplary embodiment of a calibration validation system in accordance with the present invention.

FIG. 8 illustrates an exemplary embodiment of a calibration validation method in accordance with the present invention.

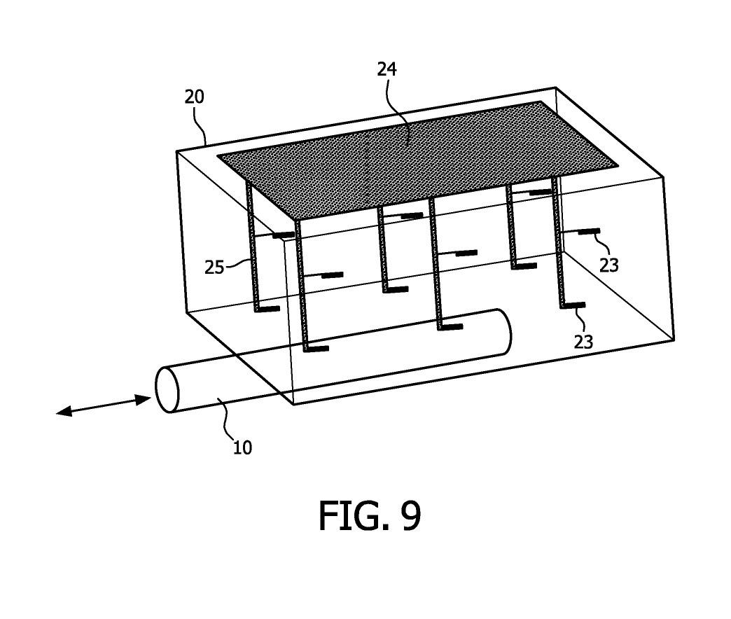

FIG. 9 illustrates an exemplary embodiment of a validation phantom in accordance with the present invention.

To facilitate an understanding of the present invention, exemplary embodiments of the present invention will be provided herein directed to an ultrasound calibration system shown in FIG. 1 and a calibration validation system shown in FIG. 7. From the description of the exemplary embodiments, those having ordinary skill in the art will appreciate how to apply the operating principles of the present invention to any type of ultrasound probe and to any type of tracking of the ultrasound probe (e.g., EM, optical, etc.).

Referring to FIG. 1, the ultrasound calibration system employs a TRUS probe 10, a calibration phantom 20, a frame assembly 21, an EM field generator 30, a EM-phantom tracker 31, an EM-probe tracker 32, and a calibration workstation 40a.

An ultrasound probe of the present invention is any device as known in the art for scanning an anatomical region of a patient via acoustic energy. An example of the ultrasound probe includes, but is not limited to, TRUS probe 10 as shown in FIG. 1.

A calibration phantom of the present invention is any type of container as known in the art of a known geometry for containing the frame assembly and having an acoustic window for facilitating a scanning of the frame assembly by the ultrasound probe. In practice, the calibration phantom may have any geometrical shape and size suitable for the calibration of one or more types of ultrasound probes. For example, as shown in FIG. 1, calibration phantom 20 generally has a prismatic shape for containing frame assembly 21 within water and/or other liquids (not shown) having sound speed equal to a sound speed in human tissue whereby TRUS probe 10 may scan frame assembly 21 from an acoustic window (not shown) below frame assembly 21.

A frame assembly of the present invention is any arrangement of one or more frames assembled within a frame coordinate system. In practice, each frame may have any geometrical shape and size, and the arrangement of the frames within the frame coordinate system is suitable for distinctive imaging by the ultrasound probe of frame pixels dependent on the relative positioning of the ultrasound probe to the calibration phantom. Examples of each frame include, but are not limited to, Z-wire frames as shown in FIGS. 3-6, N-wire frames, non-parallel frames and conically shaped frame(s).

A tracking system of the present invention is any system as known in the art employing one or more energy generator(s) for emitting energy (e.g., magnetic or optical) to one or more energy sensors within a reference area. For example, as shown in FIG. 1, EM field generator 30 emits magnetic energy to EM-phantom tracker 31 and EM-probe tracker 32 in the form of EM sensors. In an alternative embodiment, EM-phantom tracker 31 and EM-probe tracker 32 are in the form of EM field generators that emit magnetic energy to EM sensor(s) within the reference area.

The present invention is premised upon equipping the calibration phantom with one or more EM-phantom tracker(s) and upon equipping the ultrasound probe with one or more EM-probe tracker(s). In practice, the EM-phantom tracker(s) are strategically positioned relative to the calibration phantom for establishing a calibration coordinate system, and the EM-probe tracker(s) are strategically positioned relative to the calibration phantom for establishing a scan coordinate system. For example, as shown in FIG. 1, EM-phantom tracker 31 is strategically positioned on a corner of calibration phantom 20 for establishing a calibration coordinate system as symbolized thereon, and EM-probe tracker 32 is strategically positioned adjacent an ultrasound image array (not shown) of TRUS probe 10 for establishing a scan coordinate system as symbolized thereon.

The present invention is further premised on determining a transformation matrix between the frame assembly and the calibration phantom prior to the calibration of the ultrasound probe. In practice, any method as known in the art may be implemented for determining a transformation matrix between the frame assembly and the calibration phantom. For example, as related to FIG. 1, a transformation matrix T_{F→EM} between frame assembly 21 and calibration phantom 20 is derived from a mechanical registration of a frame coordinate system of frame assembly 21 (as symbolically shown thereon) to the calibration coordinate system of calibration phantom 20 during a precise manufacturing of the components.

A calibration workstation of the present invention is any type of workstation or comparable device as known in the art for controlling a calibration of the ultrasound probe in accordance with an ultrasound calibration method of the present invention. For example, as shown in FIG. 1, calibration workstation 40a employs a modular network 50a installed thereon for controlling a calibration of TRUS probe 10 in accordance with a flowchart 60 as shown in FIG. 2.

Referring to FIGS. 1 and 2, a probe localizer 51 is a structural configuration of hardware, software, firmware and/or circuitry of workstation 40a as would appreciated by those skilled in the art for localizing TRUS probe 10 within the calibration coordinate system of calibration phantom 20. More particularly, during a stage S61 of flowchart 60, probe localizer 51 receives tracking signals from EM-phantom tracker 31 and EM-probe tracker 32 to determine a coordinate position of ultrasound probe 10 within the calibration coordinate system and to compute a transformation matrix T_{P→EM} between ultrasound probe 10 and calibration phantom 20.

Ultrasound imager 52 is a structural configuration of hardware, software, firmware and/or circuitry of workstation 40a as known in the art for generating an ultrasound image of frame assembly 21 as scanned by ultrasound probe 10 during a stage S62 of flowchart 60. Based on the geometry and arrangement of frames within frame assembly 21, any particular ultrasound image of frame assembly 21 as scanned by ultrasound probe 10 will illustrate a unique spacing of frame pixels as known in the art. For example, as shown in FIG. 1, an ultrasound image 11a illustrates a spacing of frame pixels indicative of ultrasound probe 10 scanning across a midline of a pair of stacked Z-frames.

Image localizer 53 is a structural configuration of hardware, software, firmware and/or circuitry of workstation 40a as would appreciated by those skilled in the art for localizing the ultrasound image within the calibration coordinate system of calibration phantom 20. More particularly, during a stage S62 of flowchart 60, image localizer 53 processes the unique frame imaging of ultrasound image 11 (e.g., ultrasound image 11a) to determine a position of ultrasound image 11 within the frame coordinate system and to compute a transformation matrix T_{1→F} between ultrasound image 11 and frame assembly 21.

Probe calibrator 54 is a structural configuration of hardware, software, firmware and/or circuitry of workstation 40a as would appreciated by those skilled in the art for calibrating TRUS probe 10 as a function of the previously computed transformation matrixes. More particularly, during a stage S63 of flowchart 60, probe calibrator 54 executes the following equation [1] to compute a transformation matrix T_{I→T} between ultrasound probe 10 and ultrasound image 11.

T_{I→P}=(T_{P→EM})⁻¹*T_{F→EM}*T_{I→F} [1]

In practice, stages S61 and S62 may be implemented in any order or concurrently. Furthermore, flowchart 60 may be repeated as necessary or desired for different positions of the ultrasound probe relative to the calibration phantom.

To facilitate a further understanding of the ultrasound calibration system, a description of various embodiments of calibration phantom 20 and frame assembly 21 will now be provided herein.

Referring to FIG. 3, calibration phantom 20a has two (2) Z-frames 21a that create a frame coordinate system C_{F}. Calibration phantom 20a is also equipped with an EM sensor 31a that create a calibration coordinate system C_{EM}. The transformation matrix T_{F→EM} between coordinate system C_{EM} and C_{F}, is accurately known from a precise manufacturing of calibration phantom 20a.

Alternatively, calibration phantom 20a may be with up to six (6) EM sensors 31a located at precisely known location with respect to Z-frames 21a. Combined together, these sensors may be utilized to create calibration coordinate system C_{EM}, and may also be utilized for noise reduction in EM tracking. In a preferred setting, six (6) EM sensors 31a would be utilized on the side walls of calibration phantom 20.

During the calibration procedure, calibration phantom 20a is filled with water and/or appropriate liquid(s) or gels and TRUS probe 10 captures an axial image 11a of Z-frames 21a through calibration phantom 20. Z-frames 21a intersect with image 11a at six (6) points as shown in FIG. 3. The location of these intersection points can uniquely determine the location of the ultrasound image 11a within the Z-frame coordinate system C_{F}. More particularly, image localizer 23 (FIG. 1) will automatically segment the intersection points and calculate transformation matrix T_{I→F} between image coordinate system C_{I} and frame coordinate system C_{F} as known in the art.

EM sensor(s) 32a on TRUS probe 10 are localized in the calibration coordinate system C_{EM} using EM sensor(s) 31a and the EM field generator 30 such that transformation matrix T_{P→EM} between the probe coordinate system C_{P} and the calibration coordinate system C_{EM} is known. Knowing the transformation matrices T_{F→EM}, T_{P→EM} and T_{I→F}, the calibration transformation matrix T_{I→P} may be computed in accordance with equation [1] as previously described herein.

In practice, an ultrasound probe may have more than one (1) imaging array on the shaft. Typically, if there are two (2) imaging arrays, these arrays are orthogonal to each other. For example, if one array images an axial plane, then the other array images a sagittal plane. Accordingly, calibration phantom 20a may be designed and constructed to enable calibration of an axial imaging array with respect to EM sensor(s) 32a on TRUS probe 10 as shown in FIG. 3, or may be designed and constructed to enable calibration of a sagittal imaging array with respect to EM sensor(s) 32a on TRUS probe 10 as shown in FIG. 4.

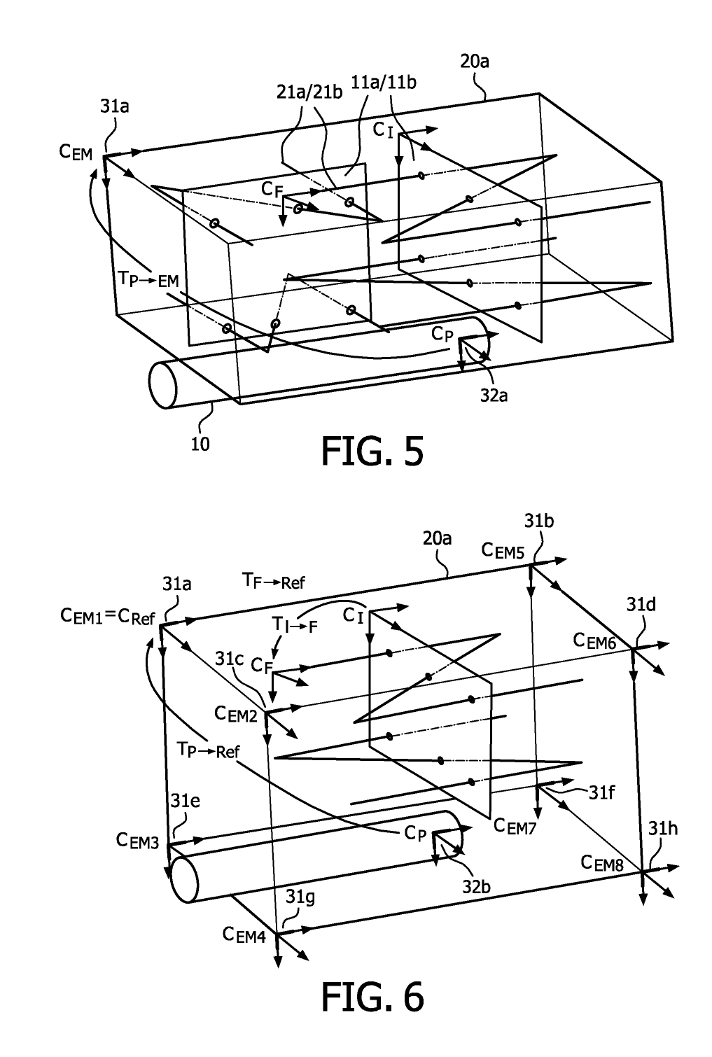

Alternatively, the two (2) imaging arrays may be simultaneously calibrated to the EM tracker on the probe by having two (2) orthogonal pairs 21a and 21b of Z-frames mounted in the calibration phantom 20 as shown in FIG. 5. In such a setup, ultrasound probe 10 may positioned such that the axial array scan an image 11a of one pair 21a of Z-structures and at the same position, the sagittal array scans an image 11b of the other pair 21b of Z-frames. This solution will not require physical movement of ultrasound probe 10 to different positions. Nonetheless, movement of probe 10 will result in calibration at different positions, which in turn results in a more accurate overall calibration.

In another embodiment (not shown in any drawing), a single pair 21a of Z-frames may be used to sequentially calibrate both the axial array and the sagittal array of ultrasound probe 10. For this embodiment, calibration phantom 20a is designed to have two (2) openings/cavities to hold ultrasound probe 10. For one opening/cavity, the axial array of ultrasound probe 10 intersects pair of Z-frames and is calibrated as previously explained herein. In the other orthogonal opening/cavity, the sagittal array of ultrasound probe 10 intersects the same pair Z-frames and is calibrated independent of the axial array calibration.

Referring back to FIG. 1, in practice, an accuracy of EM phantom trackers 31 and 32 depends on the position of EM field generator 30 since the electromagnetic field of the EM field generator 30 is not perfectly uniform. Also, any interference by metallic objects present in EM field can increase the deviations and increase the error. As EM field generator 30 may be placed in different locations between different procedures to accommodate any geometrical constraints of the reference area (e.g., an operating room), the EM tracking accuracy may be compromised.

To address the accuracy of EM phantom trackers, FIG. 6 illustrates calibration phantom 20a being equipped with eight (8) EM sensors 31 at a precisely known geometry. One of the EM sensors 31 is assumed to be a reference tracker, which may be the EM sensor the closest to the EM field generator, or the EM sensor with the smallest temporal noise. For FIG. 6, EM tracker 31a is assumed to be the reference tracker.

Accordingly, a transformation T_{EMi→Ref} from each of the other box EM trackers (C_{Emi}, i∈{2,3, . . . }) to the reference coordinate system (C_{Ref}=C_{EMI}) is known from a precise design calibration phantom 20a. In addition, there is another transformation matrix T′_{EMi→Ref} from each of EM sensor 31b-31h to reference sensor 31a measured by a tracking correction module (not shown) of calibration workstation 40a, which is different from T_{EMi→Ref} due to deviations and errors in the magnetic field inside calibration phantom 20a. Therefore a correction function ƒ may be identified in accordance with the following equation [2]:

T_{EMi→Ref}=ƒ(T′_{EMi→Ref}) [2]

where ƒ can be linear or quadratic. After identification of this corrective function, the EM measurement of the probe position is correctable by the tracking correction module of calibration workstation 40a in accordance with the following equation [3]:

T_{P→Ref}=ƒ(T′_{P→Ref}) [3]

where T′_{P→Ref} is the measured probe to reference transformation matrix by the EM tracking system and T_{P→Ref} is the corrected probe to reference transformation matrix. This new probe position delivers higher accuracy in TRUS-EM calibration.

In one scenario, the corrective function in accordance with the following equation [4]:

T_{P→Ref}=T′_{P→Ref}+Σwᵢ(xₚ, yₚ, zₚ)(T_{EMi→Ref}−T′_{EMi→Ref}) [4]

where wᵢ(xₚ,yₚ,zₚ) is a linear function and xₚ, yₚ and zₗ, are the coordinates of the TRUS probe EM-tracker measured by the tracking correction module of calibration workstation 40a.

Referring to FIG. 7, the ultrasound validation system employs TRUS probe 10, calibration phantom 20, a sensor assembly 22, EM field generator 30, EM-phantom tracker 31, EM-probe tracker 32, and a validation workstation 40b.

TRUS probe 10, calibration phantom 20, EM field generator 30, EM-phantom tracker 31 and EM-probe tracker 32 have previously described herein with reference to FIG. 1.

A sensor assembly of the present invention is any arrangement of one or more sensors (e.g., EM sensors or optical sensors) mounted within the calibration phantom. In practice, any arrangement of the sensors within the calibration phantom is suitable for positional imaging by the ultrasound probe for validation purposes of the transformation matrix between the ultrasound probe and generated images. An example of a sensor arrangement includes, but is not limited to, the EM sensors 23 as shown in FIG. 9.

A validation workstation of the present invention is workstation or comparable device as known in the art for controlling a validation of a calibration of the ultrasound probe in accordance with an ultrasound validation method of the present invention. For example, as shown in FIG. 7, a validation workstation 40b employs a modular network 50b installed thereon for controlling a validation of the calibration of TRUS probe 10 in accordance with a flowchart 70 as shown in FIG. 8.

Referring to FIGS. 7 and 8, as previously described herein, probe localizer 51 is a structural configuration of hardware, software, firmware and/or circuitry of workstation 40b as would appreciated by those skilled in the art for localizing TRUS probe 10 within the calibration coordinate system of calibration phantom 20. More particularly, during a stage S71 of flowchart 70, probe localizer 51 receives tracking signals from EM-phantom tracker 31 and EM-probe tracker 32 to determine a coordinate position of ultrasound probe 10 within the calibration coordinate system and to compute a transformation matrix T_{P→EM} between ultrasound probe 10 and calibration phantom 20.

As previously described herein ultrasound imager 52 is a structural configuration of hardware, software, firmware and/or circuitry of workstation 40b as known in the art for generating an ultrasound image of sensor assembly 22 as scanned by ultrasound probe 10 during a stage S72 of flowchart 70. Based on the arrangement of sensors within calibration phantom 20, any particular ultrasound image of sensor assembly 22 as scanned by ultrasound probe 10 will correspond to a distinctive positioning of TRUS probe 10 relative to calibration phantom 20.

Image estimator 55 is a structural configuration of hardware, software, firmware and/or circuitry of workstation 40b as would appreciated by those skilled in the art for estimating a coordinate position of each sensor illustrated in the ultrasound image based on transformation matrix T_{I→P}. More particularly, during stage S72 of flowchart 70, image estimator 55 receives tracking signals from sensor assembly 22 and estimates coordinate positions of each sensor illustrated in the ultrasound image based on transformation matrix T_{I→P} and transformation matrix T_{P→EM}.

Probe validator 54 is a structural configuration of hardware, software, firmware and/or circuitry of workstation 40b as would appreciated by those skilled in the art for visually validating the calibration of TRUS probe 10 based on the estimation of stage S72. More particularly, during a stage S73 of flowchart 70, probe validator 54 compares estimated coordinate positions of each sensor 22 within ultrasound image 12 to the actual position of each sensor 22 illustrated in the ultrasound image. For example, as shown in FIG. 7, a circular overlay represents an estimated position obtained via probe calibration process as compared to a point representing an actual position of each sensor 22 within ultrasound image 12. This provides a visual indication of the accuracy of the calibration of TRUS probe 10.

To facilitate a further understanding of the ultrasound validation system, FIG. 9 illustrates one embodiment of a sensor assembly employing a plate 24 and six (6) posts 25 downwardly extending from plate 24. Each post has two (2) EM sensors 23 attached thereto, one midway down the post and one at the end. The illustrated sensor assembly is simply placed into calibration phantom 20 whenever it is desired to validate the calibration. The illustrated sensor assembly may be designed to simultaneously be contained within calibration phantom 20 with a frame assembly 21 (FIG. 1). Also, TRUS probe 10 is mounted on a stage/stepper (not shown) that allows translational motion into and out of calibration phantom 20. The direction of the allowed motion of TRUS probe 10 is shown in FIG. 9 by the bi-directional black arrow.

In practice, validation workstation 40b (FIG. 7) may be a stand-alone workstation or incorporated within calibration workstation 40a (FIG. 1).

Referring to FIGS. 1-9, those having ordinary skill in the art will appreciate numerous benefits of the present invention including, but not limited to, an automatic calibration of an ultrasound probe.

While various embodiments of the present invention have been illustrated and described, it will be understood by those skilled in the art that the embodiments of the present invention as described herein are illustrative, and various changes and modifications may be made and equivalents may be substituted for elements thereof without departing from the true scope of the present invention. In addition, many modifications may be made to adapt the teachings of the present invention without departing from its central scope. Therefore, it is intended that the present invention not be limited to the particular embodiments disclosed as the best mode contemplated for carrying out the present invention, but that the present invention includes all embodiments falling within the scope of the appended claims.

1. An ultrasound calibration system, comprising:
a calibration phantom containing a frame assembly within a calibration coordinate system,
wherein the calibration phantom includes at least one phantom tracker establishing the calibration coordinate system;
an ultrasound probe operable to acoustically scan an image of the frame assembly within an image coordinate system relative to a scan coordinate system,
wherein the ultrasound probe includes at least one probe tracker establishing the scan coordinate system; and
a calibration workstation,
wherein the calibration workstation is operably connected to the at least one phantom tracker and the at least one probe tracker to localize the probe within the calibration coordinate system,
wherein the calibration workstation is operably connected to the at least one phantom tracker and the ultrasound probe to localize the frame assembly image within the calibration coordinate system, and
wherein, responsive to a localization of the probe and the frame assembly image within the calibration coordinate system, the calibration workstation is operable to determine a calibration transformation matrix between the image coordinate system and the scan coordinate system.

2. The ultrasound calibration system of claim 1, wherein the frame assembly mechanically registered to the calibration phantom.

3. The ultrasound calibration system of claim 1, wherein the frame assembly includes:
at least one wire frame mounted within the calibration phantom.

4. The ultrasound calibration system of claim 1, wherein the frame assembly includes:
a first set of at least one wire frame mounted within the calibration phantom; and
a second set of at least one wire frame mounted within the calibration phantom orthogonal to the first set of at least one wire frame.

5. The ultrasound calibration system of claim 4, wherein the ultrasound probe includes:
a first imaging array; and
a second imaging array orthogonal to the first imaging array.

6. The ultrasound calibration system of claim 1,
wherein the calibration phantom has a prismatic shape, and
wherein the at least one phantom tracker is attached to the calibration phantom adjacent a corner of the calibration phantom.

7. The ultrasound calibration system of claim 1,
wherein the calibration phantom has a prismatic shape, and
wherein the at least one phantom tracker is attached to at least one side wall of the calibration phantom.

8. The ultrasound calibration system of claim 1, wherein the calibration phantom includes:
a first opening for receiving the ultrasound probe; and
a second opening for receiving the ultrasound probe orthogonal to the first opening.

9. The ultrasound calibration system of claim 1, wherein the ultrasound probe is a transrectal ultrasound probe.

10. The ultrasound calibration system of claim 1,
wherein the calibration phantom includes at least one reference phantom tracker, and
wherein the calibration workstation is operably connected to the at least one phantom tracker, the at least one probe tracker and the at least one reference phantom tracker to correct for any defect in localizing the probe within the calibration coordinate system.

11. The ultrasound calibration system of claim 1, further comprising:
a sensor assembly contained within the calibration phantom,
wherein the ultrasound probe is operable to acoustically scan an image of the sensor assembly within the image coordinate system relative to the scan coordinate system,
wherein the calibration workstation is operably connected to the sensor assembly and the ultrasound probe to validate the calibration transformation matrix between the image coordinate system and the scan coordinate system.

12. The ultrasound calibration system of claim 11, wherein the sensor assembly includes:
a plate;
at least one post extending from the plate; and
at least one validation sensor attached to each post.

13. The ultrasound calibration system of claim 11, wherein the calibration workstation is operable to overlay an estimation of at least one coordinate position of the sensor assembly on a display of the image of the sensor assembly as an indication of an accuracy of the calibration transformation matrix between the image coordinate system and the scan coordinate system.

14. The ultrasound calibration system of claim 11, wherein the ultrasound probe is movable relative to the sensor assembly.

15. The ultrasound calibration system of claim 11, further comprising:
an electromagnetic field generator operable to generate an electromagnetic field at least partially encircling the at least one phantom tracker and the at least one probe tracker.

16. (canceled)

17. (canceled)

18. (canceled)

19. (canceled)

20. (canceled)
